(19) United States
(12) Patent Application Publication
(10) Pub. No.: US 2015/0275180 A1
(43) Pub. Date: Oct. 1, 2015

(54) Chemical preconditioning process for cell material to obtain chemical epigenetic reprogramming and pluripotency expression

(71) Applicant: LIPOGEMS INTERNATIONAL SRL, Milano (IT)

(72) Inventors: Carlo Tremolada, Milano (IT); Carlo Ventura, Bologna (IT)

(73) Assignee: LIPOGEMS INTERNATIONAL S.P.A., Milano (IT)

(21) Appl. No.: 14/435,423

(22) PCT Filed: Oct. 24, 2013
(86) PCT No.: PCT/IB2013/059621

§ 371 Date: Apr. 13, 2015

(30) Foreign Application Priority Data
Oct. 25, 2012 (IT) GE2012A000102

(51) Int. Cl. C12N 5/074; A61K 35/12

(52) U.S. Cl. CPC C12N 5/0696; A61K 35/12; C12N 2506/1307; C12N 2501/905

(57) ABSTRACT

Chemical preconditioning process for cell material to obtain chemical epigenetic reprogramming and pluripotency expression of stem and multipotent elements of non-embryonic origin, such as pericytes and/or mesenchymal stem cells, and/or adult somatic cells. The process includes the step of subjecting a non-expanded tissue derivative having the stem and multipotent elements, and/or of subjecting non-embryonic stem cells and/or non-embryonic somatic cells, obtained from a tissue sample or from the derivative, to a mixing with hyaluronic acid esterified with butyric and retinoic acids (HBR).

The present invention relates to a chemical preconditioning process for cell material to obtain chemical epigenetic reprogramming and pluripotency expression of stem and multipotent elements of non-embryonic origin, such as pericytes and/or mesenchymal stem cells, and/or adult somatic cells.

Experimental tests indicate that the fate of cells, and therefore also the fate of stem cells, is controlled at multiple interconnected levels, involving a complex interplay between cell signaling and epigenetic modulation that is fashioned at the level of nucleosomal assembly, the architectural establishment of multifaceted transcription factor motifs, and the temporal and spatial organization of chromatin into loops and domains.

A rapidly growing contribution to the molecular dissection of transcriptional regulation and epigenetic modifications arises from the isolation and characterization of key molecules involved in histone acetylation, DNA metylation and chromatin remodeling.

Until very recently, the fields of stem cell research and epigenetic research have been developed independently.

However, it is now clear that these fields cannot be longer viewed independently, and that the epigenetic state of toti/pluripotent stem cells may be largely responsible for the “plasticity” of these cells.

Akin to such awareness, recent findings indicate that even non-stem human adult somatic cells can be reprogrammed to an embryonic-like state by viral transduction with a few, three to four transcription factors, being transformed into lineages in which these cells would never otherwise appear.

Therefore viral vector-mediated gene delivery can be exploited to provide a proof-of-principle that transformation of somatic cells into “induced pluripotent stem cells (iPS)” can be achieved by the unlocking of chromatin domains harboring crucial architectural (epigenetic) and genetic information that may connect specialized mature cells to their own building blocks of pluripotentiality.

Nevertheless, viral vector gene delivery is a cumbersome, expensive and potentially risky approach.

The document SHIMADAHIDENORI ET AL, “Accelerated generation of human induced pluripotent stem cells with retroviral transduction and chemical inhibitors under physiological hypoxia” BIOCHEMICAL AND BIOPHYSICAL RESEARCH COMMUNICATION 13 JAN. 2012 discloses the generation of induced pluripotent stem (iPS) cells from adipose-derivated stem cells (ASCs) by the forced expression of a defined set of pluripotency-associated transcription factor, using a combination of chemical inhibitors under a setting of physiological hypoxia in conjunction with retroviral transduction of OCT4, SOX2, Klf4 and L-Myc.

Methods for dedifferentiating cells by chemical compounds are also known.

The document US 2010/0233131 discloses a cell therapeutic composition containing selenium and a method of dedifferentiating selenium-treated cells. Adipose tissue stromal cells are preferable for dedifferentiation.

It is known that the molecule composed of the hyaluronic acid esterified with butyric and retinoic acids (HBR) is able to:

(i) maximize cardiogenesis in mouse embryonic stem cells,

(ii) enhance the myocardial and vascular differentiation in human mesenchymal stem cells (hMSCs) isolated form the bone marrow, the dental pulp and fetal membranes of human term placenta,

(iii) yield preconditioning of human mesenchymal stem cells (hMSCs) to remarkably enhance hMSC-mediated cardiovascular repair in both small (rat) and large (pig) animal models of acute myocardial infarction,

(iiii) afford significant myocardial survival and repair in infarcted hearts (rats), without stem cell transplantation.

Hyaluronic acid esterified with butyric and retinoic acids (HBR) directly injected into the infarcted myocardium is able to increase histone acetylation in both the border and remote zones of the damaged tissue.

This effect is attributable to the histone deacetylase inhibitory action exerted by the butyric moiety of HBR.

In vitro experiments have confirmed a significant increase in histone H4 acetylation obtained after HBR treatment of both adult rat cardiac ventricular cardiomyocytes and Stro-1 positive rat mesenchymal stem cells, as compared with untreated cells.

In all these studies, HBR proved effective to act transcriptionally on the expression of a number of crucial genes involved in:

(i) cardiogenesis,

(ii) vasculogenesis,

(iii) cell survival,

(iiii) synthesis of stem cell releasable trophic mediators, such as vascular endothelial growth factor (VEGF) and hepatocyte growth factor (HGF), acting in a paracrine fashion as antiapoptotic, antifibrotic and angiogenic molecules.

These effects occurred at the level of both human mesenchymal stem cells (hMSCs) and Stro-1 positive rat stem cells and adult cells (for example cardiomyocytes).

The document LIONETTI VINCENZO ET AL, “Hyaluronan mixed esters of butyric and retinoic acid affording myocardial survival and repair without stem cell transplatation” THE JOURNAL OF BIOLOGICAL CHEMISTRY 26 MAR. 2010, discloses that injection of HBR into infarcted rat hearts affords substantial cardiovascular repair and recovery of myocardial performance.

Several observations have been made about hyaluronic acid (HA), about butyric acid (BU) and about retinoic acid (RA).

The document CAVALLARI GIUSEPPE ET AL “Mesenchymal stem cells and Inlet Cotransplantation in Diabetics Rats: Improved Islet Graft Revascularization and Function by Human Adipose Tissue-Derived Stem Cells Preconditioned With Natural Molecules”, CELL TRANSPLANTATION discloses the preconditioning of human ASCs with a mixture of hyaluronic, butyric and retinoic acids: hASCs exposed to the mixture are able to increase the secretion of vascular endothelial growth factor (VEGF), the transcription of angiogenic genes including VEGF, KDR and hepatocyte growth factor (HGF).

The hyaluronan receptor CD44 is highly expressed by cardiogenic cells, and normal cardiac morphogenesis is abrogated by gene silencing of hyaluronan synthase-2.

Hyaluronan fragments promote endothelial cell differentiation in a CD44-dependent manner, and CD44 plays an important role in endothelial cell function and new blood vessel formation.

Hyaluronan can be internalized via receptor-mediated endocytosis, being detected in close association with nuclear heterochromatin.

Hyaladherins (hyaluronan binding proteins) can translocate to the nucleus upon mitogenic stimulation, serving as substrates or activators for MAP kinases, or acting as vertebrate homologues of proteins involved in cell growth and differentiation.

Concerning butyrate, its histone deacetylase inhibitory action alters chromatin structure owing to the hyperacetylation of nucleosomal histones, increasing transcription factor accessibility to target cis-acting regulatory sites.

An inference of retinoic acid signaling with cardiac differentiation is supported by the finding that abnormal heart development occurs following inactivation of the RXRα gene and by combining mouse strains with mutant RAR and RXR subtypes.

Moreover, all-trans and 9-cis-retinoic acids increased the efficiency of cardiogenesis in ES mouse embryonic stem cells.

Retinoic acid plays a crucial role in mammalian vascular development, being required in endothelial cell proliferation and vascular remodeling during vasculogenesis in vivo.

In isolated nuclei, it has been provided evidence that the transcription of HBR-modulated genes was not affected by the intact molecule, but it was rather regulated by exposure to butyric and retinoic acids, being enhanced by a mixture of the two.

These findings indicate that HBR may have acted after the intracellular hydrolysis (mediated by ubiquitous esterases) of its own esterified components.

Despite these achievements, and the fact that the adipose tissue is an easily approachable source of multipotent elements with phenotypic and gene expression profiles similar to mesenchymal stem cells (hMSCs) and pericytes, the chance of clinical application of the multilineage potential of these cells is delayed by the poor/negligible cell survival within cryopreserved lipoaspirates, the difficulty of ex vivo expansion and the complexity of current Good Manufacturing Practice (cGMP) requirements for expanded cells.

Isolated and expanded cells are therefore subjected to a so called “major manipulation” and therefore they are considered as ATMPs (Advanced Therapies Medicinal Products) and are subjected to the regulation of cGMPs (current Good Manufacturing Practice).

From these observations it is therefore clear the importance, particularly for the field of regenerative medicine, of developing synthetic molecules and/or of identifying naturally occurring molecules that can replace viral vector-mediated gene delivery procedures to obtain the epigenetic reprogramming and pluripotency expression in multipotent cell elements.

Said molecules have to be able to drive stem and multipotent cells and/or non-stem somatic cells towards a pluripotent state, maximizing their differentiating properties, and their ability to secrete a number of factors acting in a paracrine fashion into a damaged tissue to evoke the recipient's healing potential.

It is also clear the importance of having a source of stem and multipotent elements of non-embryonic origin easy to be found and ready-to-use.

It is known that patent applications GE2010A000057 and WO2011/145075 describe a device and a method for preparing a tissue, particularly adipose tissue, for transplantation obtained from lobular fat extracted by liposuction.

According to the present invention it is therefore possible to use chemical substances of natural or synthetic origin for bringing back adult stem and multipotent cells or non-stem adult somatic cells to a state with characteristics similar to the embryonic one that is for optimizing the potency thereof namely for obtaining an epigenetic reprogramming and the pluripotency expression in order to use the cells for therapeutic purposes and in regenerative medicine within various clinical contexts, including the cardiovascular diseases, neurodegenerative diseases and endocrine/metabolic diseases.

To this end the invention provides the chemical preconditioning of cell material to obtain chemical epigenetic reprogramming and pluripotency expression of stem and multipotent elements of non-embryonic origin, such as pericytes and/or mesenchymal stem cells, and/or adult somatic cells, subjecting a non-expanded tissue derivative comprising said stem and multipotent elements, and/or subjecting non-embryonic stem cells and/or non-embryonic somatic cells, obtained from a tissue sample or from said derivative, to a mixing with hyaluronic acid esterified with butyric and retinoic acids (HBR).

Said substance is able to perform one or more of the following functions:
increase of histone acetylation, leading to chromatin remodeling and enhanced transcription factor accessibility to targeted chromatin domains.
increase of transcription of genes associated with stem cell pluripotentiality, such as Oct3/4, Nanog, Sox2 and other members of this family, Klf4; increase of transcription of multiple tissue-restricted genes, along with pluripotency genes, such as Mef2c, Tbx5, and genes involved in cardiogenesis, like prodynorphin, Gata4, Nkx2.5, genes involved in vasculogenesis, like VEGF, HGF, KDR, genes involved in neurogenesis, like neurogenin1, genes priming beta-pancreatic islet commitment, such as for example neurogenin 3, genes responsible for a prosurvival context within a tissue derivative, including Pim1 and Akt.

According to the present invention the use of HBR afford chromatin remodeling and epigenetic modifications within the treated cell elements even without the need of cell expansion.

The non-expanded and ready-to-use cell product, obtained by the device according to the patent with publication number WO 2011/145075, is herein defined as “Lipogems” product for simplicity reasons.

The human adipose stem cells (hASCs) obtained with said device are here called “lipogems-derived hASCs”.

Said device uses mild mechanical forces in a completely closed system, avoiding enzymes, additives and other manipulations from being used, and it is particularly advantageous in the process of epigenetic reprogramming and optimization of cell pluripotency by the use of HBR.

Differently from the lipoaspirate not treated by said device, the product obtained from the lipoaspirate by the patented device and method, so called “Lipogems” product, encompasses a remarkably preserved vascular stroma, with slit-like capillaries wedged between adipocytes and stromal stalks containing vascular channels with evident lumina. Immunohistochemistry revealed that the stromal vascular fraction (SVF) of said “Lipogems” product includes abundant cells with pericytes and/or mesenchymal stem cells (hMSC) identity which exhibit the classical commitment to osteogenic, chondrogenic and adipogenic lineages.

The device of the patent WO2011/145075 and described below allows stromal vascular niche to be preserved and it allows HBR to act on the cells encompassed into said niche.

The flow cytometry analysis of non-expanded, collagenase-treated “Lipogems” product has shown that it is composed of a significantly higher percentage of mature pericytes and mesenchymal stem cells (hMSCs) than normal lipoaspirates obtained by conventional techniques, and a lower amount of hematopoietic elements, than enzymatically-digested lipoaspirates.

Moreover differently from the conventional lipoaspirate, that is lipoaspirate not treated by the device and method of the patent WO2011/145075, the distinctive traits of “Lipogems” product freshly isolated from adipose tissue do not differ from distinctive traits of “Lipogems” product subjected to cryopreservation that is cryopreservation does not alter the biochemical characteristics of the “Lipogems” product.

It has also to be noted that the characteristics mentioned above are present also in the “Lipogems” product obtained from cadaver adipose tissue.

According to a preferred embodiment of the present invention it is provided to mix HBR with the cell product of the device and process described in WO2011/145075 (so called “Lipogems” product”) to obtain a chemical preconditioning of said product prior to its transplantation into the same donor (autologous procedure) or into a subject different from the donor (allogeneic procedure).

The chemical preconditioning has the aim of affording the chromatin remodeling and an epigenetic reprogramming of the multipotent elements encompassed within the stromal vascular fraction (SVF) of said product.

These elements are pericytes and mesenchymal stem cells hMSCs that respond to HBR by acquiring a pluripotent state, that will in turn result in the increase in commitment towards complex lineages, including myocardial, vascular (endothelial and smooth muscle), and neuronal lineages.

The epigenetic reprogramming also enhances the synthesis and secretion of multiple growth factors that will act in a paracrine fashion after transplantation of the preconditioned “Lipogems” product to impart “instructive messages” able to enhance the healing endogenous potential of the recipient damaged tissue.

Therefore the object of the present invention is an innovative process in biomedical and clinical fields since it affords, as better explained below, to obtain the epigenetic reprogramming and pluripotency expression both of multipotent elements, such as pericytes and/or mesenchymal stem cells, of non-embryonic origin and of adult somatic cells, particularly stem and multipotent elements encompassed into the stromal vascular fraction of a non-expanded tissue derivative.

These and other characteristics and advantages of the present invention will be more clear from the following description and from the annexed figures wherein:

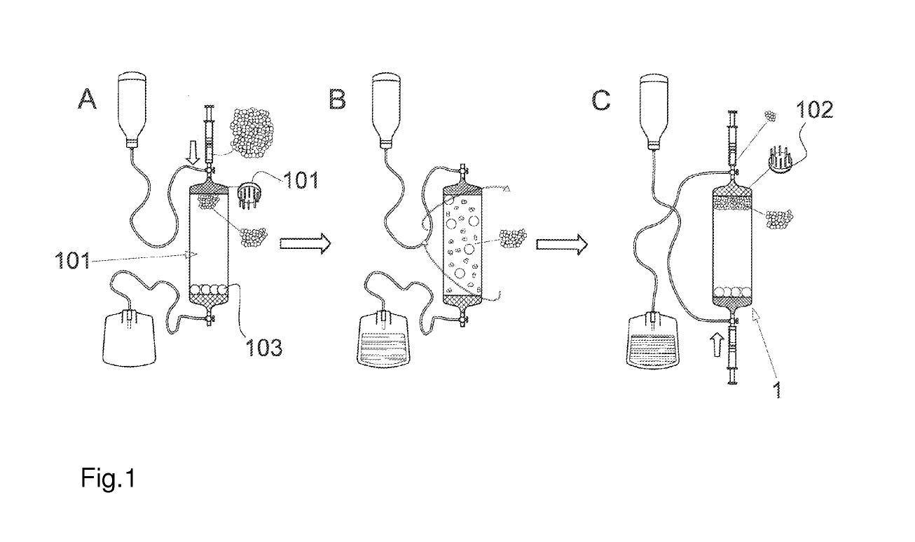

FIG. 1 schematically is the device according to the patent WO2011/145075 and the steps of treating the lipoaspirate by said device;

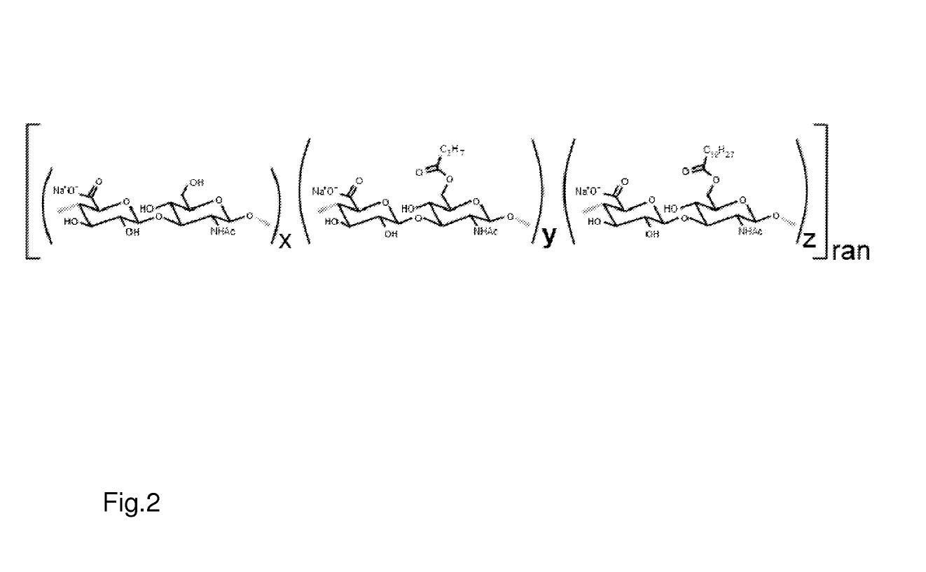

FIG. 2 is the chemical structure of HBR;

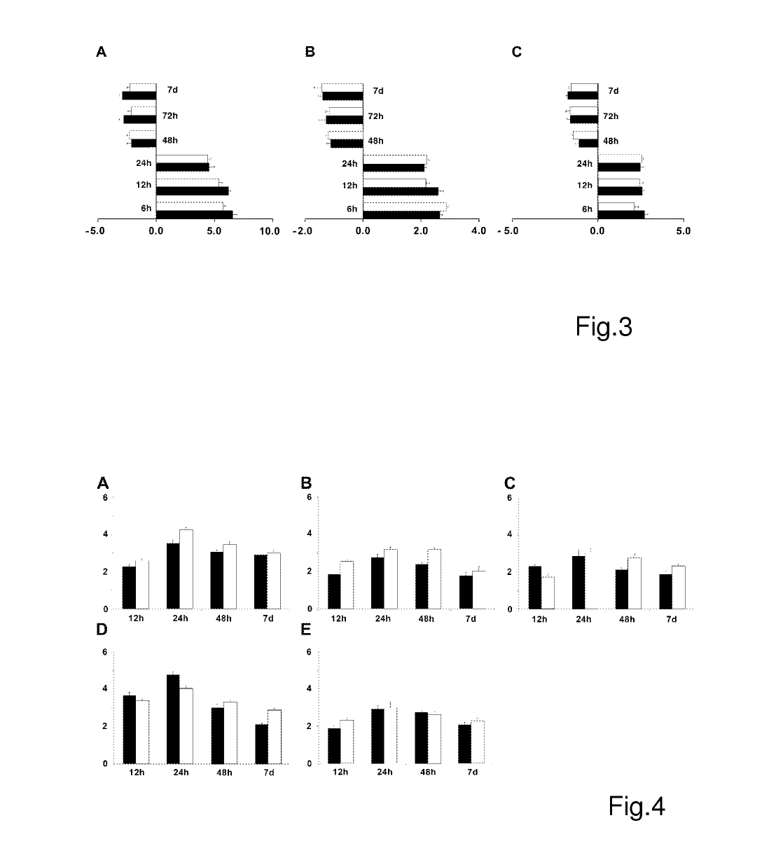

FIG. 3 is the exposure of human dermal skin fibroblasts or hASCs to HBR: Human dermal skin fibroblasts (black bars) or Lipogems-derived hASCs (white bars) were exposed for the indicated times to 2 mg/ml HBR, dissolved in phosphate buffered saline (PBS). A, Sox2; B, Oct4; C, Nanog. The amount of each transcript from exposed or unexposed cells was normalized to HPRT1, and the mRNA expression of exposed cells was plotted at each time point as fold change relative to the expression in control unexposed cells, defined as 1 (mean±S.E.; n=6);

FIG. 4 is how HBR increases the transcription of tissue restricted genes in both human dermal skin fibroblasts and hASCs such as Lipogems-derived hASCs: Human dermal skin fibroblasts (black bars) or Lipogems-derived hASCs (white bars) were exposed for the indicated times to 2 mg/ml HBR (dissolved in PBS). A, GATA4; B, Nkx2.5; C, neurogenin1; D, VEGF; E, MyoD. The amount of each transcript from exposed or unexposed cells was normalized to HPRT1, and the mRNA expression of exposed cells was plotted at each time point as fold change relative to the expression in control unexposed cells, defined as 1 (mean±S.E.; n=6);

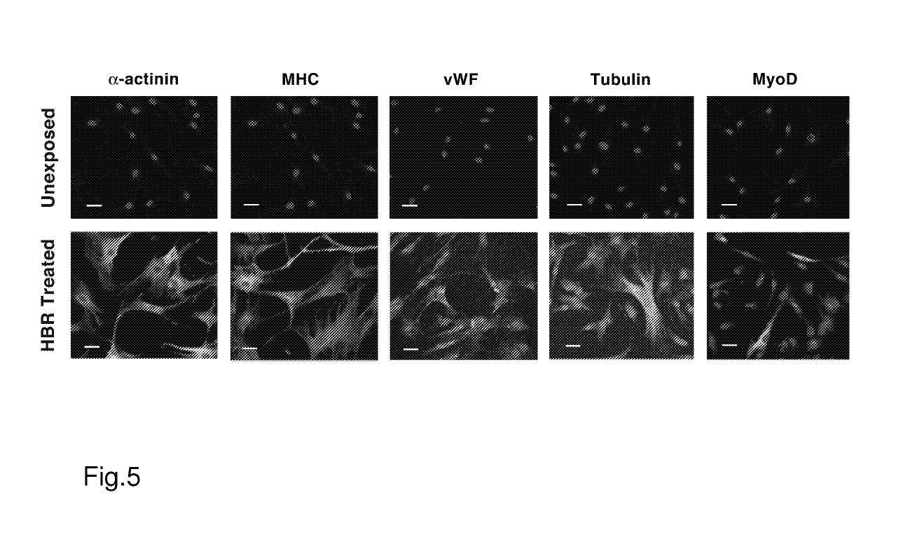

FIG. 5 is how the exposure of human dermal skin fibroblasts to HBR triggers the commitment towards multiple lineages: Confocal microscopy analysis revealed that, after exposure to HBR (2 mg/ml, in PBS) for 14 days, human adult non-stem cells were reprogrammed to express tissue restricted markers of terminal commitment, including α-sarcometic actinin (α-actinin), and α-myosin heavy chain (MHC) for cardiac differentiation, von Willebrand Factor (vWF) for endothelial differentiation, β-3 Tubulin (Tubulin) for neuronal commitment, and myoD for skeletal myogenesis. Scale bars are 40 μm. Representative of six separate experiments;

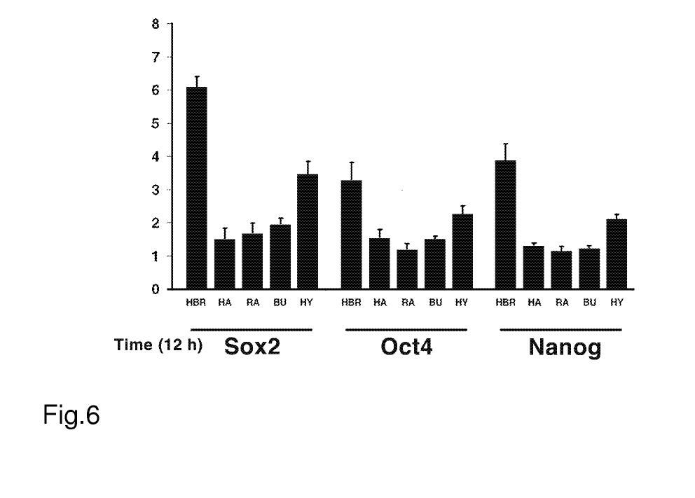

FIG. 6 is the comparative analysis of the effect of HBR, or its grafting moieties alone or as a mixture, on the expression of sternness-related genes in human dermal skin fibroblasts: cells were incubated for 12 hours in the absence or presence of 2.0 mg/ml HBR, 1.5 mg/ml HA, 2.5 mM BU, or 10-8M RA, or exposed to a hydrolyzed (HY) HBR solution (2 mg/ml), obtained from a 2-h basic HBR hydrolysis followed by pH neutralization. This procedure affords a complete release of each single HBR grafted moiety, leading to the formation of a mixture of HA, BU, and RA, with each molecule in the concentration range reported above for the experiments based on cell incubation in the presence of each molecule alone. The amount of each transcript from exposed or unexposed cells was normalized to HPRT1, and the mRNA expression of exposed cells was plotted at each time point as fold change relative to the expression in control unexposed cells, defined as 1 (mean±S.E.; n=6).

The synthesis and characterization of the HBR compound can be obtained according to known procedures such as those described in the following documents:
Ventura C, Maioli M, Asara Y, Santoni D, Scarlata I, Cantoni S, Perbellini A. Butyric and retinoic mixed ester of hyaluronan. A novel differentiating glycoconjugate affording a high throughput of cardiogenesis in embryonic stem cells. J Biol Chem. 2004 May 28; 279(22):23574-9.
Lionetti V, Cantoni S, Cavallini C, Bianchi F, Valente S, Frascari I, Olivi E, Aquaro G D, Bonavita F, Scarlata I, Maioli M, Vaccari V, Tassinari R, Bartoli A, Recchia F A, Pasquinelli G, Ventura C. Hyaluronan mixed esters of butyric and retinoic acid affording myocardial survival and repair without stem cell transplantation. J Biol Chem. 2010 Mar. 26; 285(13):9949-61.

With reference to FIG. 2, the abbreviation HA means hyaluronic acid; the abbreviation BU means butyric acid, the abbreviation RA means retinoic acid.

The general formula of HA esters represents a random copolymer (ran) of three distinct dimeric repeating units, among which x are non-substituted, y are butyrylated (C₃H₇CO group) and z are retinoylated (C₁₉H₂₇CO group).

Let n be the sum of x, y, and z, namely the total number of disaccharide units in the polysaccharide.

DS_{BU} and DS_{RA} correspond to the ratio between y and n and between z and n respectively.

Obviously, for hyaluronic (H)-retinoic (R) monoester, abbreviated as HR, the DS_{BU} is 0 (y=0), whereas for hyaluronic (H)-butyric (B) monoester, abbreviated as HB, DS_{RA} is 0 (x=0).

According to a known preparation method, the primary hydroxyl group in position 6 of the N-acetyl-D-glucosamine residues in the polysaccharide backbone is the most reactive towards esterification.

A double salt of tetrabutylammonium with two functional groups of hyaluronan, specifically its carboxyl and 6-hydroxyl has been prepared, in order to achieve a good solubility in polar aprotic organic solvents and to increase the nucleophilicity of the oxygen atom at C-6.

Retinoylation with retinoyl chloride, which is the factor limiting the reaction rate, was carried out before butyrylation by means of butyric anhydride and 4-(dimethylamino)pyridine as a hypernucleophilic acylation catalyst.

The degree of substitution (DS) was considered as the number of the esterified OH groups for each repeating unit of hyaluronic acid (GlcNAc-GlcUA dimer).

The weight-average molecular weight of HBR, referred to as the weight-average of sodium hyaluronate, was determined by high performance size exclusion chromatography.

All of the synthesized HBRs may exhibit a DS with BU (DSBU) ranging between 0.05 and 1.5, or any other DSBU that may result compatible with the stability, and biological efficiency/efficacy of HBR.

The DS with RA (DSRA) may be between 0.002 and 0.1, or any other DSRA that may result compatible with the stability, and biological efficiency/efficacy of HBR.

The DSBU/DSRA ratio may be 6, or any other value that may result compatible with the stability, and biological efficiency/efficacy of HBR.

The weight average molecular weight may be ranging between 10,000 and 30,000 daltons, or other values that may result compatible with chemical stability, and biological efficiency/efficacy of HBR.

The HBR can be used for a direct reprogramming of non-stem somatic cells or optimization of multipotency/differentiating potential of adult stem cells towards complex lineages, including the commitment to cardiac, vascular, neuronal, and skeletal muscle fates. In fact:
The hyaluronan receptor CD44 is highly expressed by cardiogenic cells, and normal cardiac morphogenesis is abrogated by disruption of hyaluronan synthase-2;
Hyaluronan fragments promote endothelial cell differentiation in a CD44-dependent manner, and CD44 plays an important role in endothelial cell function and new blood vessel formation;
Hyaluronan can be internalized via receptor-mediated endocytosis, being detected in close association with nuclear heterochromatin. Hyaladherins (hyaluronan binding proteins) can translocate to the nucleus upon mitogenic stimulation, serving as substrates or activators for MAP kinases, or acting as vertebrate homologues of proteins involved in cell growth and differentiation. Therefore, hyaluronan that is taken up may regulate (stem) cell lineage commitment from within subcellular compartments, and may also act as a carrier for internalization of hyaluronan-grafted molecules, such as retinoic and butyric acids;
Butyrate, exhibits histone deacetylase inhibition, and therefore alters chromatin structure owing to the hyperacetylation of nucleosomal histones, increasing transcription factor accessibility to target cis-acting regulatory sites. Accordingly, in mouse ES (embryonic stem) cells inhibition of histone deacetylase by trichostatin A and shear stress-induced hystone acetylation afford epigenetic modifications leading to the transcriptional activation of VEGF receptor 2 promoter, and other gene expression signatures of both endothelial and cardiac lineage commitment;
Retinoic acid, based on its recruitment of R-X-R receptor superfamilies, elicits signaling pathways leading to both cardiogenic and neurogenic decisions. Retinoic acid also plays a crucial role in mammalian vascular development, being required in endothelial cell proliferation and vascular remodeling during vasculogenesis in vivo. Within this context, histone deacetylase inhibitors enhance RXR/RAR heterodimer action, promoting crucial developmental pathways in pluripotent cells.

The “multicomponent-multitarget” logics of HBR is also supported by the finding that intriguing interplay(s) occur between cardiogenic and neurogenic transcription factors, creating an interconnected molecular plight for both cardiogenesis and neurogenesis. MASH-1 and other bHLH transcription factors are essential for neurogenic commitment and are upregulated by myocyte enhancer factor 2C (Mef2C). Mef2C is early expressed during neurogenesis, but it is also crucial for cardiogenic commitment in embryonic stem (ES) and multipotent cells. Gene transduction with Mef2C, together with GATA4 and Nkx2.5, respectively encoding a zinc finger and homeodomain essential for cardiogenesis in different animal species, including Humans, is mandatory in direct reprogramming of skin fibroblasts to cardiac lineage, without an induced pluripotent stem cell (iPS) intermediate. Nkx2.5, besides acting as a cardiogenic orchestrator, is also remarkable for neuronal differentiation in both skeletal muscle and ES cells. Intriguingly, endorphin peptides that display a significant role in neurogenesis were found to act as major conductors of cardiogenesis in ES cells.

HBR triggers an early activation of sternness related genes in both human dermal skin fibroblasts, and human adipose stem cells (hASCs).

In non-stem adult somatic cells, such as human dermal skin fibroblasts, and in multipotent adult stem cells, such as hASCs obtained with the device disclosed in the patent WO 2011/145075, called lipogems-derived hASCs, HBR exerted a biphasic effect on the expression of sternness-related genes, including Nanog, Sox2, and Oct4. In both cell types, during a 7-day treatment, the mixed ester significantly enhanced the transcription of these genes within the first 12-24 hours, then inducing a down-regulation at later times of exposure (FIG. 3).

Moreover HBR enhances the transcription of cardiogenic, neurogenic, vasculogenic and skeletal-myogenic genes in both human dermal skin fibroblasts, and hASCs, such as Lipogems-derived hASCs.

In both cell types, during a 7-day treatment, HBR significantly enhanced the gene expression of GATA4, Nkx2.5 (cardiogenesis), Vascular Endothelial Growth Factor (VEGF) (vasculogenesis), neurogenin-1 (neurogenesis), and myoD (skeletal myogenesis) (FIG. 2). The transcriptional response was remarkably significant already after 12 hours of exposure, peaking at 24 hours, and remaining stable afterwards (FIG. 4).

Moreover HBR induces the expression of tissue restricted marker proteins in both human dermal skin fibroblasts, and hASCs, such as Lipogems-derived hASCs.

Confocal microscopy analysis provided evidence that exposure of both cell types to HBR for 14 days resulted in the appearance of cells expressing:
Cardiac specific markers, such as α-myosin heavy chain, and α-sarcomeric actinin;
The neural specific protein β-3 tubulin;
The endothelial marker vonWillebrand factor (vWF);
The skeletal muscle marker myoD;

FIG. 5 shows as an example the multilineage commitment of human dermal skin fibroblasts following a 14-day treatment with HBR.

Therefore, HBR was even able to promote the terminal commitment of human non-stem somatic cells to lineages in which these cells would never otherwise appear (FIG. 5).

The use of HBR has many advantages, as compared with the use of its individual grafting moieties alone, or as a mixture.

In the FIG. 6 it is shown that HBR enhanced the transcription of each gene over the control value at significantly higher levels when compared to the transcriptional response elicited by HA, BU or RA alone, or as a mixture obtained from HBR hydrolysis.

To perform a comparative analysis between the reprogramming effect of HBR, hyaluronic acid (HA), butyric acid (BU), or retinoic acid (RA), alone or in combination, human skin fibroblasts or Lipogems-derived hASCs were incubated for 12 hours in the absence or presence of 2.0 mg/ml HBR, 1.5 mg/ml HA, 2.5 mM BU, or 10-8M RA, or exposed to hydrolyzed HBR obtained from a 2-hour basic HBR hydrolysis followed by pH neutralization. This procedure affords a complete release of each single HBR grafted moiety, leading to the formation of a mixture of HA, BU, and RA, with each molecule in the concentration range reported above for the experiments based on (stem) cell incubation in the presence of each molecule alone. FIG. 6 shows as an example that exposure of human skin fibroblasts to BU, RA, or HA alone significantly increased the transcription of the stemness/pluripotency genes Sox2, Nanog and Oct4. The transcriptional responses were further enhanced following exposure to hydrolyzed HBR, resulting in the release of all moieties grafted within the mixed ester HBR (FIG. 6). However, under these experimental conditions, the gene transcription was considerably lower than that detected in the presence of HBR (FIG. 6), indicating a substantial advantage in the use of the mixed ester HBR, as compared to the use of its grafted moieties, considered individually or as a mixture. Hence, a maximal efficiency in direct reprogramming of non-stem somatic cells or in the expression of the differentiating potential of adult multipotent stem cells can be achieved when HA, BU, and RA are concomitantly internalized by the mixed ester, suggesting that this compound may have provided optimal intracellular HA/BU/RA ratios, and/or timely action, probably subsequent to the hydrolysis of the mixed ester by ubiquitous intracellular esterases.

There are advantages in the use of HBR mediated-reprogramming over viral vector-induced reprogramming of somatic or stem cells.

HBR, after inducing an early increase in the expression of Nanog, Sox2 and Oct4, led to a downregulation of these sternness genes after 24 hours of treatment. These transcription factors are master regulators, silencing genes that are waiting to create the next generation of cells, and upon their disabling stem cells rapidly begin to differentiate. Ablation of the Oct4 gene in mouse embryos prevented proliferation of inner cell mass (ICM) cells and promoted differentiation into trophectoderm. Once expressed, Nanog blocks differentiation. Thus, negative regulation of Nanog is required to sustain differentiation during ES cell commitment. Similarly, early phases of ES cell differentiation involved a down-regulation of Sox2 expression.

A number of interrelated observations provide a clear advantage in the use of HBR for direct reprogramming of human adult somatic cells or human adult multipotent stem cells to multiple lineages that had long been pursued as major target commitments for regenerative medicine (i.e. cardiac, vascular, neural and skeletal muscle lineages):
Viral vector-mediated transduction of human skin fibroblasts with Sox2, Oct4, and Nanog is a cumbersome procedure requiring extensive (stem) cell manipulation;
Persistent activation of these genes in engineered cells is associated with very low differentiation efficiencies (32). This poor outcome severely hampers the use of viral vector mediated gene deliver as a tool for translating (stem) cell reprogramming into a clinical practice;
The low efficiency of differentiation in viral vector transduced cells raises cautionary issues concerning the fate of the large proportion of cells that remain undifferentiated after commitment to defined lineages. In particular, it is now evident that stray cells that haven't fully differentiated might have the ability to turn into an unwanted cell type, like a tumor or a cell that just doesn't fulfill the desired requirement(s) for a targeted tissue repair.

On the contrary, HBR initially switched to pluripotency/multipotency optimization both human adults stem cells and human adult non-stem somatic cells without freezing cells into an iPS-like intermediate state, thus avoiding a decrease in differentiation efficiency from a long-term overexpression of sternness-related genes.

Worthy to note, flow cytometry analysis performed in human skin fibroblasts exposed for 14 days to 2 mg/ml HBR revealed that 12.30±2.7% of cells expressed β-3-tubulin (neuronal determination marker), 18.0±2.6 of cells expressed vWF (endothelial marker), whereas the percentage of myoD (skeletal muscle)- and α-sarcomeric actinin (myocardial)-positive elements was 14.00±3.00%, or 28.00±3.40%, respectively (mean±S.E.; n=6). Under the same experimental conditions, exposure of Lipogems-derived hASCs to HBR yielded 14.0±2.1% of β-3-tubulin expressing cells, 22.2±3.0% of vWF positive cells, 12.4±2.0% of myoD positive cells, and 31.0±2.8% of cells expressing α-sarcomeric actinin (mean±S.E.; n=6).

The device 1 described in the patent WO 2011/145075 allows tissue to be taken from human or animal alive patient or cadaver, to be processed without using enzymes, possibly to cryopreserve it, and to re-inject it in the patient, it being possible for said patient to be the same or different than the donour patient (autologous or allogeneic transplantation).

The device 1 allows a non-expanded tissue derivative to be prepared from a non-enzymatic “minimal manipulation” of the original tissue.

Preferably the treated tissue is adipose tissue.

According to a particularly advantageous embodiment, the tissue comprises adipose tissue obtained from lobular fat material extracted, for example, by liposuction, said fat material being composed of a fluid component comprising an oil component, a hematic component and/or sterile solutions and of a solid component comprising vascular-stromal structures, cell fragments and/or one or more cell macroagglomerates of heterogeneous size and comprising stem cells.

In the present disclosure tissue derivative means a cell aggregate or cluster, comprising a vascular-stromal fraction enriched with stem and multipotent elements, such as pericytes and/or mesenchymal stem cells.

Preferably in the present invention the non-expanded tissue derivative is obtained from adipose tissue treated by the method and device 1 described below and in the document WO 2011/145075.

The process for preparing the tissue derivative from fat material provides the step of dividing said fat material into cell agglomerates with a smaller size than the size of said macroagglomerates, such that said cell and/or vascular-stromal agglomerates have a size equal to or smaller than a predetermined value, and such that said sizes are on average equal to one another.

As an alternative or in combination with the division step, the process can provide at least one step for washing the cell aggregates which is carried out contemporaneously with a step separating the fluid component from the solid component.

According to the present invention the step of subjecting said cells/agglomerates to a mixing with one or more chemical substances of natural or synthetic origin is carried out for all the length of the process or only for a part thereof or at the end of the process.

Obviously it is possible to provide to obtain a tissue derivative also by means of other known methods and devices.

The device 1, by means of at least one net 101 reducing the size of the lipoaspirate and of mechanical stirring elements 103, that allow an emulsion of the liquid components of the lipoaspirate to be obtained, progressively reduces the size of the cell clusters or agglomerates of the lipoaspirated adipose tissue, contemporaneously removing the liquid residues mainly composed of oil and blood.

The method for treating the lipoaspirate provides to wash and to reduce the sizes of the cell clusters under a complete immersion into a liquid, such as a washing sterile physiological solution, such to minimize the traumatic action of the device 1 on the cells.

The reducing, washing and emulsion steps carried out mechanically by the device 1 therefore are performed in the absence of air inside said device 1.

The device 1 allows cell agglomerates with smaller sizes to be obtained which improve the post-transplantation cell integration.

The first reduction of cell agglomerates is obtained by pushing the lipoaspirate contained into the suction syringe into the device 1 through the first size reducing net 101, while a corresponding amount of saline solution is discharged from the device 1 and is collected into a collection bag (FIG. 1A).

Once the desired amount of lipoaspirate has been inserted in the device 1, maintained in a vertical position, with the first size reducing net 101 at the top, the floating layer of adipose tissue, preferably, has not to occupy more than the upper half of the device 1.

The stirring elements 103 provided in the device 1, for example balls made of metal or the like, when stirring the device 1 (FIG. 1B) allow an emulsion between oil, blood and washing solution to be made which is removed against density, from inside the device 1, following the flow of the saline solution moved by gravity, while the agglomerates with a reduced size of the stromal vascular fraction (containing adipocytes, vessels, pericytes and mesenchymal cells) move towards the upper part of the device 1 considered in the vertical position (FIG. 1B).

When the liquid part inside the device 1 is clear and the lipoaspirate is yellow, the flow of saline solution is stopped, and the device 1 is rotated by 180° (FIG. 1C).

The second reduction of the size of the cell clusters is obtained by passing the agglomerates of the stromal vascular fraction (containing adipocytes, vessels, pericytes and mesenchymal cells) through a second size reducing net 102 by pushing inside the device 1 additional liquid through the lower aperture of the device (considered in the vertical position as in FIG. 1C), by using for example a syringe.

The tissue derivative obtained by treating the lipoaspirate with the device 1 described above is collected into syringes connected to the upper aperture of the device (considered in the vertical position as in FIG. 1C) and it is ready to be used or preserved.

The product obtained by means of the device described above therefore is a non-expanded tissue derivative comprising a stromal vascular fraction enriched with stem and multipotent elements, such as pericytes and/or mesenchymal stem cells particularly it is a “natural stromal vascular niche” encompassing a network of scaffolding-like adipocytes entangled with highly preserved vessels, containing a remarkable yield of pericytes and mesenchymal stem cells.

According to the present invention the cell material on which the chemical preconditioning can be carried out comprises, as an alternative or in combination to one another:
stem and multipotent elements, such as pericytes and/or mesenchymal stem cells, contained in the stromal vascular fraction of an expanded or non-expanded tissue derivative;
stem and multipotent elements, such as pericytes and/or mesenchymal stem cells obtained from said derivative or from a tissue sample, expanded or non-expanded,
non-stem adult somatic cells (e.g. skin fibroblasts) contained or obtained from said tissue derivative or sample, expanded or non-expanded.

Therefore the method can be applied also to cells different than stem and multipotent cells, such as human or animal adult somatic cells, or non human embryonic cells to obtain a kind of programming thereof.

The method can be applied both on cell material obtained from alive donours or from cadaveric donors.

Moreover it can be also applied, in addition to fresh material, on material thawed after cryopreservation at −80° C. or in liquid nitrogen.

It is noted that non-expanded tissue derivative means an aggregate of cells of the tissue taken from a patient, which is not cultured and therefore the cell (stem and non-stem) elements contained therein are not cultured in vitro into a culture medium, that is they are not subjected to proliferation (defined also as expansion) in vitro.

As a whole, the process can comprise the steps of:

a) preparing a non-expanded tissue derivative from non-enzymatic “minimal manipulation”, of the original tissue, such as lipoaspirate, said derivative being intended as composed of aggregates of cells of the original tissue, such as adipocytes in case of lipoaspirate, encompassed by a vascular-stromal component containing stem cells and/or multipotent elements such as pericytes and mesenchymal stem cells;

b) as an alternative or in combination to step a), preparing a cell suspension from a tissue sample or from the derivative as of step a), collecting the (non-embryonic) stem cells and/or the adult somatic cells from said cell suspension;

c) subjecting said (non embryonic) stem cells and/or the adult somatic cells of the tissue derivative or obtained from a tissue sample or from the derivative as of step a), to mixing with hyaluronic acid esterified with butyric and retinoic acids (HBR).

Said HBR can be mixed alone or in combination with other liquid and/or solid substances.

The term “minimal manipulation” of cells means that (stem and non-stem) cells are not expanded (cultured and proliferated in vitro in culture) and are not subjected to a series of “treatments” such as enzymatic dissociations and extractions, centrifugations and separations of cell populations, enriching of some cell populations to the detrimental of other ones (for example flow cytofluorometry separation) and other similar treatments.

The fact that cells can be subjected to a minimal manipulation allows the process and the product obtained by said process not to fall within the “Drug-major manipulation” regulations.

Obviously it is also possible to provide the cell elements of non-embryonic origin (stem and multipotent elements and somatic cells) to be subjected to expansion in vitro.

The tissue sample can advantageously comprise adipose material extracted for example by liposuction/lipoaspiration, and the step b) of the process can provide the adipose material to be enzymatically treated for releasing multipotent and stem elements and/or somatic cells after possibly having reduced the adipose tissue into smaller parts.

Therefore the process can be applied both to non-expanded cell aggregates (for example cells obtained by the process described in the patent WO2011/145075), and to cells derived therefrom (and therefore expanded cells) or other types of stem cells (of non-embryonic origin) and non-stem cells (for example adult somatic cells such as fibroblasts) that could be isolated and expanded.

Said isolated and expanded cells are therefore subjected to a so called “major manipulation” and are considered as ATMPs (Advanced Therapies Medicinal Products) and are subjected to the regulation of cGMPs (current Good Manufacturing Practice).

Stem and multipotent cells (of non-embryonic origin) and/or somatic cells to be treated can be obtained by any known methods.

Preferably the stem and multipotent cells and/or the somatic cells to be treated according to the method of the present invention are contained into a tissue derivative obtained by the method and the device described in the international patent published under the number WO 2011/145075.

According to one embodiment, the container wherein there is provided the cell material to be treated is the one used in the device for preparing the adipose tissue for transplantation described in the international patent application published with the number WO2011/145075. In practice it is a container made of sterile glass or plastic, or anyway a translucid material, preferably resistant to high temperatures and processable in autoclave, inside which the lipoaspirated adipose material is injected.

By the treatment method, according to the present invention, for the tissue derivative defined also in the present patent application as “Lipogems” product obtained by the method and device of the patent WO2011/145075 it is possible to obtain not only adipose material to be used as a biologic filler, but also material rich in elements such as pericytes and mesenchymal stem cells with an enriched potency or anyway a modified potency, that can be used for regenerating tissues even different than those from which said material has been extracted.

The chemical preconditioning to obtain epigenetic reprogramming and transcriptional modulation, according to the modes of the present invention, can be performed not only, preferably, on a non-expanded tissue derivative such as that obtained by the method and the device of the patent WO2011/145075, but also on specific types of cells.

These types of cells can be:
all types of cells derived from the so called “Lipogems” product, in any way isolated and expanded in culture for any period of time; these cell types may be pericytes, as well as adipose derived stem cells contained within the stromal vascular fraction (SVF) of the “Lipogems” product, or any other cell type provided in the “Lipogems” product and/or its stromal vascular fraction SVF.
non-stem adult somatic cells, including human fibroblasts isolated from the skin and other sources (for example scarred tissues); in human dermal skin fibroblasts, HBR is able to afford the transcriptional expression of sternness related genes, including Oct3/4, Nanog, Sox2 and other members of this family, Klf4.

Therefore according to the present invention the chemical preconditioning implies, also for somatic cells, the acquirement of a pluripotent state with the possibility of driving the cell differentiation to any type of cell lineages derived from the three germ layers.

Particularly the chemical reprogramming of fibroblasts (that is somatic cells) into induced pluripotent stem cells is an unprecedented strategy for tissue healing.

According to the present invention the chemical preconditioning to obtain a substantial epigenetic reprogramming and a transcriptional modulation in stem and pluripotent cells of non-embryonic origin or in adult somatic cells, occurs by mixing the cell material with hyaluronic acid esterified with butyric and retinoic acids (HBR).

Said HBR is provided alone or in a mixture, in combination with other solid and/or liquid substances.

HBR is mixed within a wide range of doses, preferably but not exclusively ranging from 0.5 to 2.5 mg/l.

Preferably therefore the doses can vary, from 0.5 to 2.5 mg/ml.

The mixing with the cell material has to be carried out gently.

The incubation time of the preconditioned cell material may be very short, ranging between 1 to 2 hours, or less, as the effect of the drug will continue after transplantation of the cell material on the patient.

The mixing has a length preferably ranging from 1 to 2 hours, it being modulatable on the basis of the optimization of the differentiation to particular specific lineages.

The molecule composed of the hyaluronic acid esterified with butyric and retinoic acids (HBR) can be used dissolved in physiological solution or buffers, or it may be subjected to encapsulation into “smart nanoparticles/nanocolloidal containers”, i.e. self-assembled biodegradable multilayer nanoshells or nanofilms, produced by the so-called Layer-by-Layer (LbL) technique, or any other nano-structured entity that may be suitable for this purpose, to afford efficient release at both intracellular and tissue levels.

Preferably the cell material mixed with HBR is composed of the non-expanded tissue derivative obtained by the device and method described above and object of the patent with publication number WO2011/145075, defined in the present patent application as “Lipogems” product.

The reprogramming effect involves:
increase of histone acetylation, leading to chromatin remodeling and enhanced transcription factor accessibility to targeted chromatin domains;
increase of transcription of genes associated with stem cell pluripotentiality (i.e. Oct3/4, Nanog, Sox2 and other members of this family, Klf4);
increase of transcription of multiple tissue-restricted genes, along with pluripotency genes, including, Mef2c, Tbx5, and:
genes involved in cardiogenesis, like prodynorphin, Gata4, Nkx2.5,
genes involved in vasculogenesis, like VEGF, HGF, KDR,
genes involved in neurogenesis, like neurogenin1
genes priming beta-pancreatic islet commitment, such as neurogenin 3,
genes responsible for a prosurvival context within the “Lipogems” product, including Pim1 and Akt.

The vasculogenic and prosurvival actions elicited by HBR, together with the antifibrotic effects provided by some of the HBR-induced growth factors (i.e. VEGF and HGF), result in a favorable healing outcome in any damaged tissue suffering from ischemic conditions and scar formation, in any way arising from the most various contexts, including atherosclerotic diseases or diabetes complications.

It is worthy to note that the preconditioned tissue derivative, particularly the “Lipogems” product, homes within the site of injection, and that its vascular bed forming the stromal vascular fraction (SVF) establishes anastomoses and connections with the recipient's vasculature, leading to progressive engraftment. This in turn results in a progressive cross-talk among the stem elements of the stromal vascular fraction (i.e. pericytes and mesenchymal stem cells) and the host environment. In particular, such a cross-talk will involve the stromal vascular fraction delivery of long-lived signals for cell survival, reperfusion, angiogenesis, and cell pluripotency that will “clear” the hostile environment of the damaged recipient tissue.

The role of HBR within this context is crucial. In fact, its persistence after the initial incubation within the transplanted tissue derivative and its subsequent internalization into the cellular elements of said derivative, via the hyaluronan CD44 receptor (highly expressed in pericytes and mesenchymal stem cells of the stromal vascular fraction), leads to the intracellular release of hyaluronan grafted moieties, followed by hyaluronan-mediated priming of transcription factor/protein kinase interaction (essential for nuclear translocation and trafficking), butyric acid-mediated inhibition of histone deacetylases and chromatin remodeling, and retinoic acid-mediated transcriptional modulation (i.e. influencing all the above mentioned gene expression patterning).

HBR driven expression of pluripotentiality and lineage commitment within the stromal vascular fraction of the tissue derivative, such as that of the tissue derivative defined as “Lipogems” has a favorable impact with the recipient tissue dynamics since:
a larger number of elements of the stromal vascular fraction are committed to vascular, or myocardial, or neural lineages, or they provide enhanced release of trophic mediators with angiogenic, antiapoptotic and antifibrotic properties;
the progressive engraftment of the tissue derivative, particularly of the “Lipogems” product results in an entanglement of rescuing signals from the product itself and the recipient, promoting a mutual survival cell patterning.
the establishment of mutual survival circuitries is the prerequisite for survival of elements committed to cell lineages featured for the substitution or repair of the lost cellular type or types within the recipient's damaged tissue.

The use of HBR on cell material, particularly on the “Lipogems” product, allows an optimal recovery of any type of damaged tissue to be obtained.

The preconditioned cell material can be used not only for autologous transplantations but also for transplantations in allogeneic setting.

Particularly the non-expanded human “Lipogems” product has been subjected to xenogeneic transplantation in rats subjected to chronic hindlimb ischemia.

The transplantation was devoid of side effects, it was well tolerated by the recipient rats.

After 14 days, no signs of immune rejection, nor inflammatory infiltrates were observed at both physical and histological examination.

The xenogeneic transplantation of human “Lipogems” product affords efficient revascularization and functional recovery in rats subjected to chronic hindlimb ischemia, even by using the cryopreserved Lipogems product. Besides revascularization it is noted the maintenance of an optimal muscle trophism, which results to be dependent, at histological examination, from a neoformation of muscle fibres, likely starting from satellite cells of the skeletal muscle.

The experimental results indicate that the non-expanded tissue derivative defined in the present invention “Lipogems”, fresh or cryopreserved, subjected to epigenetic reprogramming and transcription modulation chemically by the modes of the present invention also in allogeneic transplantations, has not led to immune rejection, nor it led to local or systemic inflammatory outcomes, and it induced clear tissue revascularization and repair together with normalization of its function.

Due to such effects the “Lipogems” cell product is a new and innovative product in the field of cell therapy and regenerative medicine.

Therefore the present invention relates also to cell material particularly non-embryonic stem and/or multipotent cells obtainable by a process as described above and to the use of said cells for preparing a drug for regenerating and/or repairing an animal or human tissue to be used for therapeutic purposes and in regenerative medicine for allogeneic or autologous transplants.

The present invention relates also to the use of hyaluronic acid esterified with butyric and retinoic acids (HBR) for preparing a drug comprising non-embryonic multipotent and stem elements and/or non-embryonic somatic cells for regenerating and/or repairing an animal or human tissue to be used for therapeutic purposes and in regenerative medicine for allogeneic or autologous transplants.

HBR can be provided alone or mixed with other solid and/or liquid substances.

Obviously the invention is not limited to the embodiments described above but it can be widely modified.

For example it is possible to provide to chemically precondition cells not only inside a container but also in vivo that is directly on the patient to be treated such to vary the potency thereof already inside the tissue where they are provided.

In this case HBR or the compound containing HBR is applied directly on or in the area to be treated.

The treatment of a tissue, particularly a damaged tissue directly on the patient provides one or more of the following steps:

a) clearing the hostile fibrotic, ischemic and apoptotic tissue environment associated to the inherent disease condition by administering, for example by injecting into the tissue hyaluronic acid esterified with butyric and retinoic acids (HBR) to obtain the chemical epigenetic reprogramming and pluripotency expression of stem and multipotent elements of non-embryonic origin, such as pericytes and/or mesenchymal stem cells, and/or adult somatic cells

b) transplanting, under allogeneic or autologous conditions, cell material preconditioned according to the present invention.

Preferably the transplantation is carried out with the non-expanded tissue derivative defined as “Lipogems” preconditioned ex vivo (1-2 hours or less) with HBR.

The cell material preconditioned and used for the transplantation, particularly the preconditioned “Lipogems” product, can be prepared as fresh or originally cryopreserved.

The cell material preconditioned and used for the transplantation, particularly the preconditioned “Lipogems” product, can be obtained from human or animal alive donors or from cadavers.

The cell material preconditioned and used for the transplantation, particularly the preconditioned “Lipogems” product, can be used for autologous or allogeneic use.

c) administering in the cell material transplanted according to step b) and/or in the surrounding tissue hyaluronic acid esterified with butyric and retinoic acids (HBR) to obtain chemical epigenetic reprogramming and pluripotency expression of stem and multipotent elements of non-embryonic origin, such as pericytes and/or mesenchymal stem cells, and/or adult somatic cells. As described above HBR can be used alone or in a mixture with other solid and/or liquid substances.

Step c) is a post-transplant optimization in vivo of the tissue response and/or the rescuing potential of the multipotent elements contained within the stromal vascular fraction SFV of the transplanted “Lipogems” product by injection in the tissue of HBR.

On the whole, the pre- and post-transplant injection of HBR, will enhance the regenerative potential of the material used for the transplantation, particularly according to one embodiment of the present invention, it will enhance the regenerative potential associated to the preconditioned “Lipogems” product, reinforcing the signaling cross-talk between the transplanted product and the recipient tissue, maximizing the action of HBR possibly released from the preconditioned “Lipogems” product itself.

It has been demonstrated that the tissue rescue is related to the ability of HBR to enhance the capillary density and myocardial perfusion, to promote the recruitment of endogenous mesenchymal stem cells to the site of damage, to decrease the number of apoptotic cardiomyocytes, to remarkably decrease the extent of damaged tissues. These beneficial effects were mediated at the transcriptional level.

According to the present invention therefore it is possible to obtain chemically not only the preconditioning, that is an epigenetic reprogramming and a transcriptional modulation, of cells, tissue derivatives and/or tissues to be used for autologous or allogeneic transplantations but also the preconditioning of the recipient tissue with clear beneficial effects on the healing process.

### REFERENCES

1. Cherry A B, Daley G Q. Reprogramming cellular identity for regenerative medicine. Cell. 2012 Mar. 16; 148(6):1110-22.
2. Ang Y S, Gaspar-Maia A, Lemischka I R, Bernstein E. Stem cells and reprogramming: breaking the epigenetic barrier? Trends Pharmacol Sci. 2011 July; 32(7):394-401.
3. Barrero M J, Izpisua Belmonte J C. Regenerating the epigenome. EMBO Rep. 2011 March; 12(3):208-15.
4. Shafa M, Krawetz R, Rancourt D E. Returning to the stem state: epigenetics of recapitulating pre-differentiation chromatin structure. Bioessays. 2010 September; 32(9):791-9.
5. Takahashi K, Tanabe K, Ohnuki M, Narita M, Ichisaka T, Tomoda K, Yamanaka S. Induction of pluripotent stem cells from adult human fibroblasts by defined factors. Cell. 2007 Nov. 30; 131(5):861-72.
6. Ventura C, Maioli M, Asara Y, Santoni D, Scarlata I, Cantoni S, Perbellini A. Butyric and retinoic mixed ester of hyaluronan. A novel differentiating glycoconjugate affording a high throughput of cardiogenesis in embryonic stem cells. J Biol Chem. 2004 May 28; 279(22):23574-9.
7. Ventura C, Cantoni S, Bianchi F, Lionetti V, Cavallini C, Scarlata I, Foroni L, Maioli M, Bonsi L, Alviano F, Fossati V, Bagnara G P, Pasquinelli G, Recchia F A, Perbellini A. Hyaluronan mixed esters of butyric and retinoic Acid drive cardiac and endothelial fate in term placenta human mesenchymal stem cells and enhance cardiac repair in infarcted rat hearts. J Biol Chem. 2007 May 11; 282(19):14243-52.
8. Simioniuc A, Campan M, Lionetti V, Marinelli M, Aquaro G D, Cavallini C, Valente S, Di Silvestre D, Cantoni S, Bernini F, Simi C, Pardini S, Mauri P, Neglia D, Ventura C, Pasquinelli G, Recchia F A. Placental stem cells pre-treated with a hyaluronan mixed ester of butyric and retinoic acid to cure infarcted pig hearts: a multimodal study. Cardiovasc Res. 2011 Jun. 1; 90(3):546-56.
9. Lionetti V, Cantoni S, Cavallini C, Bianchi F, Valente S, Frascari I, Olivi E, Aquaro G D, Bonavita F, Scarlata I, Maioli M, Vaccari V, Tassinari R, Bartoli A, Recchia F A, Pasquinelli G, Ventura C. Hyaluronan mixed esters of butyric and retinoic acid affording myocardial survival and repair without stem cell transplantation. J Biol Chem. 2010 Mar. 26; 285(13):9949-61.
10. Wheatley S C, Isacke C M, Crossley P H. Restricted expression of the hyaluronan receptor, CD44, during postimplantation mouse embryogenesis suggests key roles in tissue formation and patterning. Development. 1993 October; 119(2):295-306.
11. Camenisch T D, Spicer A P, Brehm-Gibson T, Biesterfeldt J, Augustine M L, Calabro A Jr, Kubalak S, Klewer S E, McDonald J A. Disruption of hyaluronan synthase-2 abrogates normal cardiac morphogenesis and hyaluronan-mediated transformation of epithelium to mesenchyme. J Clin Invest. 2000 August; 106(3):349-60.
12. Takahashi Y, Li L, Kamiryo M, Asteriou T, Moustakas A, Yamashita H, Heldin P. Hyaluronan fragments induce endothelial cell differentiation in a CD44- and CXCL1/GRO1-dependent manner. J Biol Chem. 2005 Jun. 24; 280(25):24195-204.
13. Savani R C, Cao G, Pooler P M, Zaman A, Zhou Z, DeLisser H M. Differential involvement of the hyaluronan (HA) receptors CD44 and receptor for HA-mediated motility in endothelial cell function and angiogenesis. J Biol Chem. 2001 Sep. 28; 276(39):36770-8.
14. Tammi R, Rilla K, Pienimaki J P, MacCallum D K, Hogg M, Luukkonen M, Hascall V C, Tammi M. Hyaluronan enters keratinocytes by a novel endocytic route for catabolism. J Biol Chem. 2001 Sep. 14; 276(37):35111-22.
15. Majumdar M, Meenakshi J, Goswami S K, Datta K. Hyaluronan binding protein 1 (HABP1)/C1QBP/p32 is an endogenous substrate for MAP kinase and is translocated to the nucleus upon mitogenic stimulation. Biochem Biophys Res Commun. 2002 Mar. 8; 291(4):829-37.
16. Grammatikakis N, Grammatikakis A, Yoneda M, Yu Q, Banerjee S D, Toole B P. A novel glycosaminoglycan-binding protein is the vertebrate homologue of the cell cycle control protein, Cdc37. J Biol Chem. 1995 Jul. 7; 270(27):16198-205.
17. Wolffe A P, Pruss D. Targeting chromatin disruption: Transcription regulators that acetylate histones. Cell. 1996 Mar. 22; 84(6):817-9.
18. Illi B, Scopece A, Nanni S, Farsetti A, Morgante L, Biglioli P, Capogrossi M C, Gaetano C. Epigenetic histone modification and cardiovascular lineage programming in mouse embryonic stem cells exposed to laminar shear stress. Circ Res. 2005 Mar. 18; 96 (5):501-8.
19. Kastner P, Grondona J M, Mark M, Gansmuller A, LeMeur M, Decimo D, Vonesch J L, Done P, Chambon P. Genetic analysis of RXR alpha developmental function: convergence of RXR and RAR signaling pathways in heart and eye morphogenesis. Cell. 1994 Sep. 23; 78(6):987-1003.
20. Sucov H M, Dyson E, Gumeringer C L, Price J, Chien K R, Evans R M. RXR alpha mutant mice establish a genetic basis for vitamin A signaling in heart morphogenesis. Genes Dev. 1994 May 1; 8(9):1007-18.
21. Wobus A M, Kaomei G, Shan J, Wellner M C, Rohwedel J, Ji Guanju, Fleischmann B, Katus H A, Hescheler J, Franz W M. Retinoic acid accelerates embryonic stem cell-derived cardiac differentiation and enhances development of ventricular cardiomyocytes. J Mol Cell Cardiol. 1997 June; 29(6):1525-39.
22. Lai L, Bohnsack B L, Niederreither K, Hirschi K K. Retinoic acid regulates endothelial cell proliferation during vasculogenesis. Development. 2003 December; 130(26):6465-74.
23. Dilworth F J, Fromental-Ramain C, Yamamoto K, Chambon P. ATP-driven chromatin remodeling activity and histone acetyltransferases act sequentially during transactivation by RAR/RXR In vitro. Mol Cell. 2000 November; 6(5):1049-58.
24. Cavallari G, Olivi E, Bianchi F, Neri F, Foroni L, Valente S, Manna G L, Nardo B, Stefoni S, Ventura C. MESENCHYMAL STEM CELLS AND ISLET COTRANSPLANTATION IN DIABETIC RATS: IMPROVED ISLET GRAFT REVASCULARIZATION AND FUNCTION BY HUMAN ADIPOSE TISSUE-DERIVED STEM CELLS PRECONDITIONED WITH NATURAL MOLECULES. Cell Transplant. 2012 Apr. 2. [Epub ahead of print].

The invention claimed is:

1. A chemical preconditioning process for cell material to obtain chemical epigenetic reprogramming and pluripotency expression of stem and multipotent elements of non-embryonic origin, such as pericytes and/or mesenchymal stem cells, or adult somatic cells, comprising the step of:
subjecting a non-expanded tissue derivative comprising said stem and multipotent elements, or subjecting one of both of non-embryonic stem cells or non-embryonic somatic cells, obtained from a tissue sample or from said derivative, to a mixing with hyaluronic acid esterified with butyric and retinoic acids (HBR).

2. The chemical preconditioning process according to claim 1, further comprising the step of having the hyaluronic acid esterified with butyric and retinoic acids (HBR) perform one or more of the following functions:
increasing histone acetylation, thereby leading to chromatin remodeling and enhanced transcription factor accessibility to targeted chromatin domains;
increasing transcription of genes associated to stem cell pluripotency; or
increasing transcription of multiple tissue-restricted genes, along with pluripotency genes, genes involved in cardiogenesis, genes involved in neurogenesis, genes priming beta-pancreatic islet commitment, or genes responsible for a pro-survival context within a tissue derivative.

3. The chemical preconditioning process according to claim 1, wherein the step of mixing with hyaluronic acid esterified with butyric and retinoic acids (HBR) comprises mixing in combination with other solid or liquid substances.

4. The chemical preconditioning process according to claim 1, wherein the hyaluronic acid esterified with butyric and retinoic acids (HBR) is mixed within a range of doses from 0.5 to 2.5 mg/l.

5. The chemical preconditioning process according to claim 1, wherein the step of mixing the non-expanded tissue derivative and the hyaluronic acid esterified with butyric and retinoic acids (HBR) has a length ranging from 1 to 2 hours, the step of mixing being modulated based on optimization of differentiation to particular specific lineages.

6. The chemical preconditioning process according to claim 1, wherein the hyaluronic acid esterified with butyric and retinoic acids (HBR) is dissolved in a physiological solution or a buffer solution, or is subjected to encapsulation into biological containers configured to achieve an effective release of the hyaluronic acid esterified with butyric and retinoic acids (HBR) both at intracellular level and at tissue level.

7. The chemical preconditioning process according to claim 1, further comprising the steps of:
(a) preparing a non-expanded tissue derivative from non-enzymatical minimal manipulation of the original tissue, as a lipoaspirate, said tissue derivative comprising aggregates of cells of an original tissue having a vascular-stromal component containing stem cells or multipotent elements;
(b) in alternative to or in combination to step (a), preparing a cell suspension from a tissue sample or from the tissue derivative of step (a), and collecting one of both the stem cells or adult somatic cells from said cell suspension; and
(c) subjecting one or both of said stem cells or adult somatic cells of the tissue derivative, or obtained from a tissue sample, or from the derivative processed according to step (a), to a mixing with the hyaluronic acid esterified with butyric and retinoic acids (HBR).

8. The chemical preconditioning process according to claim 7, wherein the original tissue comprises fat material, step (b) of the preconditioning process providing the fat material to be enzymatically treated for releasing one or both of the stem cells or the somatic cells after optionally reducing adipose tissue into smaller portions.

9. The chemical preconditioning process according to claim 7, wherein the tissue derivative comprises transplantation adipose tissue obtained from lobular fat material, said lobular fat material having a fluid component comprising an oil component, a hematic component or sterile solutions, and further having a solid component comprising vascular-stromal structures, cell fragments, or one or more cell macroagglomerates of heterogeneous size, and further comprising stem cells,
the process comprising the step of dividing said lobular fat material into cell agglomerates of a smaller size than a size of said macroagglomerates, such that said cell or vascular-stromal component has a size equal to or smaller than a predetermined value, and such that sizes of the vascular-stromal components are on average equal to one another, the step (c) being carried out for an entire length of the process or only for a part thereof or at an end of the process.

10. The chemical preconditioning process according to claim 9, further comprising, as an alternative to or in combination with the step of dividing, the step of washing the cell aggregates which is carried out contemporaneously with a step of separating the fluid component from the solid component, the step (c) being carried out for all the length of the process, or only for a part thereof, or at the end of the process.

11. Non-embryonic stem or multipotent cells obtained by a process according to claim 1.

12. The chemical preconditioning process according to claim 1, further comprising the step of preparing a drug using the preconditioned cell material for regenerating or repairing an animal or human tissue for therapeutic purposes and in regenerative medicine for allogeneic or autologous transplants.

13. A method of preparing a drug comprising non-embryonic multipotent and stem cells or non-embryonic somatic cells for regenerating or repairing an animal or human tissue, the method comprising:
providing a non-expanded tissue derivative comprising said non-embryonic multipotent and stem cells or non-embryonic somatic cells;
mixing said non-expanded tissue derivative with hyaluronic acid esterified with butyric and retinoic acids (HBR); and
preparing a drug therewith having therapeutic and regenerative properties for allogeneic or autologous transplants.

14. A process of treating a tissue for construction or regenerative purpose on a patient, comprising:
(a) clearing a hostile tissue environment associated to a disease condition by administering, in the tissue, hyaluronic acid esterified with butyric and retinoic acids (HBR) to obtain the chemical epigenetic reprogramming and pluripotency expression of stem and multipotent elements of non-embryonic origin, the hyaluronic acid esterified with butyric and retinoic acids (HBR) being provided alone or in combination with other solid or liquid substances;
(b) transplanting, under allogeneic or autologous conditions, cell material preconditioned according to claim 1; and
(c) administering, in the cell material transplanted according to step (b) or in surrounding tissue, the hyaluronic acid esterified with butyric and retinoic acids (HBR) to obtain chemical epigenetic reprogramming and pluripotency expression of stem and multipotent elements of non-embryonic origin, or adult somatic cells, the hyaluronic acid esterified with butyric and retinoic acids (HBR) being provided alone or in combination with other solid or liquid substances.
